(19) United States
(12) Patent Application Publication
(10) Pub. No.: US 2023/0241005 A1
(43) Pub. Date: Aug. 3, 2023

(54) Encapsulation of cannabinoids

(71) Applicant: Phagelux (Canada) Inc., Montreal (CA)

(72) Inventors: Nancy Tawil, Cantley (CA); Tristan Lépine, Laval (CA)

(21) Appl. No.: 18/160,263

(22) Filed: Jan. 26, 2023

Related U.S. Application Data
(60) Provisional application No. 63/304,122, filed on Jan. 28, 2022.

(51) Int. Cl. A61K 31/05; A61K 9/50; A61K 9/00; A61K 9/51; A61K 47/36; A61K 47/46

(52) U.S. Cl. CPC A61K 31/05; A61K 9/5031; A61K 9/0014; A61K 9/5123; A61K 47/36; A61K 47/46

(57) ABSTRACT

Cannabidiol (CBD) is the major nonpsychotropic constituent of cannabis, having numerous applications in the management of clinical conditions, including the treatment of chronic pain. The delivery of CBD and other cannabinoids is hindered by poor bioavailability, low aqueous solubility, and instability in the solution form, hepatic first pass effect, enzymatic degradation in the gut, and mucociliary clearance. We propose the encapsulation of cananbinoids in biodegradable polyester amide urea polymer, and in similar polymers, for the optimized prolonged topical delivery and permeability of CBD for pain, inflammation and other conditions.

### FIELD OF THE INVENTION

The present invention relates to the general field of conditions including inflammation, among others, and pain treatment and is more particularly concerned with encapsulation of cannabinoids for the transdermal delivery, for example for delivery of cannabidiol (CBD) and other similar molecules.

### BACKGROUND

Cannabidiol (CBD) is the major nonpsychotropic constituent of cannabis, having numerous applications in the management of clinical conditions, including the treatment of chronic pain. CBD can serve as a viable replacement or adjunct to non-steroidal anti-inflammatory drugs and opiates as well as the management of anorexia, side-effects of chemotherapy, multiple sclerosis, migraine, muscle spasms, schizophrenia, and epilepsy.

Delivery of CBD has been associated with major drawbacks, such as poor bioavailability, low aqueous solubility, instability in the solution form, hepatic first pass effect, enzymatic degradation in the gut, and mucociliary clearance. The identified limitations of conventional routes of administration emphasize the need for evaluating other routes for more effective and sustained therapeutic delivery of CBD.

Accordingly, there exists also a need for new methods and media for delivering cannabinoids, including CBD and other similar compounds. An object of the present invention is to provide such methods and media.

### SUMMARY OF THE INVENTION

In a broad aspect, there is provided a method of delivering at least one cannabinoid to a subject, the method comprising applying on a skin of the subject a composition including biodegradable capsules, wherein the biodegradable capsules include a solution of the at least one cannabinoid encapsulated in a hydrophobic polymer.

There may also be provided a method wherein the at least one cannabinoid is provided to treat a condition selected from the group consisting of seizures associated with Lennox-Gastaut syndrome or Dravet syndrome, arthritis, chronic pain, anorexia, side-effects of chemotherapy, multiple sclerosis, migraine, muscle spasms, schizophrenia, epilepsy, and a dermatological condition.

There may also be provided a method wherein the dermatological condition is selected from acne vulgaris, dermatitis, psoriasis, hidradenitis suppurativa, Kaposi Sarcoma, and pruritus.

There may also be provided a method wherein the at least one cannabidoid includes cannabidiol (CBD), and wherein the composition releases from about 5 mg to about 50 mg of CBD over a duration of between 12 and 72 hours.

There may also be provided a method wherein 15 mg of CBD is released in about 48 hours.

There may also be provided a method wherein at least 10% of the CBD contained in the composition is delivered transdermally.

In another broad aspect, there is provided a composition comprising at least one cannabinoid in solution encapsulated in hollow biodegradable polymeric capsules.

In another broad aspect, there is provided a composition comprising biodegradable polymeric capsules, the biodegradable polymeric capsules including a first phase encapsulated in a second phase surrounding the first phase, wherein the first phase includes at least one cannabinoid and the second phase includes a biodegradable polymer.

There may also be provided a composition wherein the biodegradable polymeric capsules include at least one of nanocapsules and microcapsules.

There may also be provided a composition wherein the biodegradable polymeric capsules include a polymer selected from
(1) a poly (ester amide urea) wherein at least one diol, at least one diacid, and at least one amino acid are linked together through an ester bond, an amide bond, and a urea bond,
(2) a poly (ester urethane urea) wherein at least one diol and at least one amino acid are linked together through an ester bond, a urethane bond, and a urea bond,
(3) a poly (ester amide urethane urea) wherein at least one diol, at least one diacid, and at least one amino acid are linked together through an ester bond, an amide bond, a urethane bond, and a urea bond,
(4) a poly (ester amide urethane) wherein at least one diol, at least one diacid, and at least one amino acid are linked together through an ester bond, an amide bond, and a urethane bond,
(5) a poly (ester urea) wherein at least one diol and at least one amino acid are linked together through an ester bond and a urea bond, and
(6) a poly (ester urethane) wherein at least one diol and at least one amino acid are linked together through an ester bond and a urethane bond, further wherein
the at least one diol is a compound of formula:
HO—R₁—OH, R₁ is chosen from C₂-C₁₂ alkylene optionally interrupted by at least one
oxygen, C₃-C₅ cycloalkylene, C₃-C₁₀ cycloalkylalkylene,

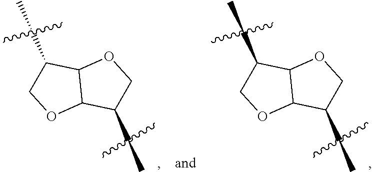 [SMILES: *[C@@H]1COC2C1OC[C@@H]2* ; *[C@@H]1COC2C1OC[C@H]2*]

the at least one diacid is a compound of formula:
HO—(CO)—R₃—(CO)—OH, R₃ is C₂-C₁₂ alkylene,
   the at least one amino acid is chosen from naturally occurring amino acids and non-naturally occurring amino acid.

There may also be provided a composition wherein the polymer is a poly (ester amide urea) comprising the following two blocks with random distribution thereof:

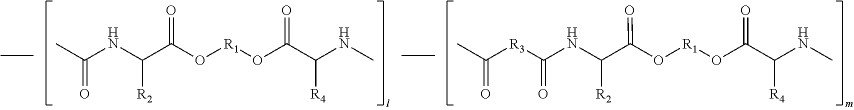

wherein

the ratio of 1:m ranges from 0.05:0.95 to 0.95:0.05, 1+m=1, R₁ is chosen from C₂-C₁₂ alkylenes optionally interrupted by at least one oxygen, C₃-C₈ cycloalkylenes. C₃-C₁₀ cycloalkvlalkylenes,

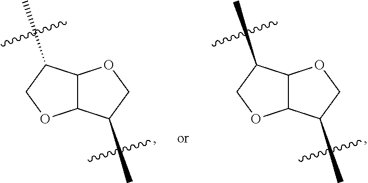

R₃ is C₂-C₁₂ alkylene,
R₂ and R₄ are independently chosen from the side chains of L- and D- amino acids so that the carbon to which R₂ or R₄ is attached has L or D chirality.

There may also be provided a composition wherein R₁ is —(CH₂)₆—, R₃ is —(CH₂)₈—, and both R₂ and R₄ are the side chain of L-leucine.

There may also be provided a composition further comprising a permeation enhancer.

There may also be provided a composition wherein the permeation enhancer is selected from the group consisting of hydrocarbons, including alkanes, alkenes, squalene, mineral oil, halogens, alcohols, including glycerols, glycols, polyglycols, ethanol, caprylic alcohol, laury alcohol, organic acids, including oleic acid, undecanoic acid, amines, amides, including pyrrolidone(N-methyl-2-pyrrolidone 2-pyrrolidon)azones, (1-dodecylazacycloheptan-2-one))urea, isopropyl myristate, sodium lauryl sulfate, cetyltrimethyl ammonium bromide, sorbitan monolaurate, polysorbate 80, dodecyl dimethyl ammoniopropane sulfate, dimethyl sulfoxide, dodecyl methyl sulfoxide, phospholipids, water (for example 70:30, v/v), and carbopol.

There may also be provided a composition wherein the formulation is for administration across at least one of dermal and epidermal barriers, and wherein the capsules are configured so as to not enter systemic circulation.

There may also be provided a composition wherein the at least one cannabinoid is derived from at least one of cannabis concentrates, isolated cannabinoids and cannabis extracts.

There may also be provided a composition wherein the at least one cannabinoid is selected from the group consisting of: phytocannabinoids, synthetic cannabiboids, and cannabimimetics.

There may also be provided a composition further comprising hyaluronic acid.

There may also be provided a composition further comprising collagen.

There may also be provided a composition wherein the at least one cannabinoid is cannabigerol (CBG).

There may also be provided a composition wherein the at least one cannabinoid is cannabidiol (CBD).

There may also be provided a composition wherein the capsules include nanocapsules having a diameter of between 10 nm and 1 μm.

There may also be provided a composition wherein the nanocapsules are between 100 nm and 1 μm in diameter.

There may also be provided a composition wherein the capsules include microparticles having a diameter of between 1 μm and 250 μm.

There may also be provided a composition wherein the first phase includes the at least one cannabinoid in a solution, the solution including the at least one cannabinoid, a solvent and an emulsifier.

There may also be provided a composition wherein the emulsifier includes Kolliphor P188.

There may also be provided a composition wherein the solvent is an oil.

There may also be provided a composition further comprising Tween 80.

Advantageously, the proposed composition and method can deliver relatively large quantities of cannabinoids after topical application with a relatively long release period. Typically, the cannabinoid is in solution in a solvent, such as non-limitingly an edible oil, and is in such embodiments in the form of a cannabinoid oil. An emulsifier, such as non-limitingly Kolliphor P188, is added in some embodiments to the cannabinoid oil to allow preparation using the double emulsion process. It was found using this process that a major portion of the cannabinoid can be retained in a liquid phase that is encapsulated in a solid phase, a hollow solid capsule. This is highly surprising given the solubility of CBD in organic solvents.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

In the drawings:

FIG. 1 illustrates the particle size distribution of the composition obtained from example 1;

FIG. 2A, in a scanning electron microscopy image, illustrates the shape of the capsules obtained from example 1;

FIG. 2B, in a scanning electron microscopy image, illustrates in greater details the shape of the capsules obtained from example 1.

### DETAILED DESCRIPTION

A method of topical and/or transdermal delivery of cannabinoids, such as CBD, is proposed. Topical and transdermal delivery offer multiple advantages over other routes of administration. The proposed method allows in some embodiments for the delivery of CBD and other cannabinoids in the management of diverse conditions in user-friendly dosage forms that ensure sustained and consistent delivery of efficient doses in the management of medical conditions for which they are found effective. Moreover, topical and transdermal dosage forms promote adherence and eliminate the need for frequent drug consumption.

Without being binding nor restricting the scope of the appended claims, topical and transdermal delivery of CBD is supported by the presence of multiple receptors expressed on the skin, including, but not limited to, CB1 and CB2 receptors and transient receptor potential (TRP). CB1 and CB2 receptors are involved in regulating the immune response, diminishing inflammatory responses by inhibiting the release of pro-inflammatory mediators and through immune cell modulation. Cannabinoids also modulate several noncannabinoid receptors and ion channels and act through various receptor-independent pathways, e.g. delaying the reuptake of endocannabinoids and neurotransmitters and by enhancing or inhibiting the binding action of certain G-protein-coupled receptors. These receptors permit CBD regulation of cellular proliferation, differentiation, and apoptosis as well as cytokine activity in the skin, providing a scientific rationale for the potential use of CBD and other cannabinoids in various dermatological conditions including acne vulgaris, dermatitis, psoriasis, hidradenitis suppurativa, Kaposi Sarcoma, and pruritus.

Transdermal delivery may be formulated with a favorable release kinetics, enabling controlled drug delivery with clinically relevant drug concentration in skin layers and sustained systemic delivery of CBD for an intermediate delivery length, lasting for example days to weeks in chronic cases or conditions warranting long term treatment. Several studies have provided insight on the efficacy of applying CBD topically for the management of pain and inflammation. In particular, Lodzki et al. investigated the effectiveness of transdermal application of CBD for the management of inflammation in rheumatoid arthritis. Results show a time dependent reduction of inflammation in a murine models. However, a lag in the onset of effect can be seen due to the lack of fast skin permeation. The delivery vehicle is therefore in some embodiments formulated to provide an immediate therapeutic effect in addition to prolonged release of transdermal CBD for pain and inflammation. Hamell et al., showed effectiveness of transdermal CBD in a rat model for arthritis, where significant suppression of inflammatory markers and pain reduction were shown following the application of a CBD containing gel. A dose-dependent effect was shown with optimal reduction of inflammation seen with a 6.2 mg/day dosing regimen.

The present document proposes a unique suit-to-purpose technology that allows for the sustained release of cannabionoids. For example, CBD, or any other suitable cannabinoid, is encapsulated in capsules, such as mircocapsules or nanocapsules. These capsules are biodegradable, and contain in their center a solution including the cannabinoid, or pure cannabinoid. For example, in the specific case of nanocapsules incorporating CBD in biodegradable nanocapsuless, many potential benefits can be achieved in some embodiments. The nanoencapsulation technology increases the stability, bioavailability and offers a controlled release of CBD to the different skin layers. In a specific embodiment, the nanocapsules are made of amino-acid biodegradable polyester amide urea (PEAU) co-polymer which is entirely composed on non-toxic building blocks of readily available monomers comprising two moles of alpha amino acids and one mole of diols. Other similar polymers are also usable in alternative embodiments. These compounds contain hydrolysable ester bonds at a monomer stage, which when incorporated into the polymeric backbones are responsible for the biodegradation of the polymer. These hydrolysable ester bonds are carefully monitored to achieve sustained, controlled release, while the mechanical properties of the polymer are given by amide functions. In some embodiments, the product is dispensed in a dosage-metered spray bottle, or roll-on applicator, with each dose allowing for a controlled release of from about 5 to about 50 mg of CBD for a duration of between 12 and 72 hours, for example 15 mg of CBD for up to 48 hours. Other dosages and release kinetics are also within the scope of the invention. Example of parameters that are used in some embodiments are:
ranges for nanoparticle size for transdermal: 10 nm to 1 μm;
ranges for microparticle size for topical: 1 μm to 250 μm;
ranges for dosage: 5 mg to 200 mg
ranges for time delivery: 12 hours to 5 days.
   However, other values for these parameters are usable.

The proposed amino acid based biopolymers are easy to produce, have low cost, are stable, do not cause immunogenic reactions and are biodegradable. The present invention is based on encapsulated cannabinoids in microcapsules or nanocapsules, or combinations thereof, made of a suitable polymer, such as a polyester amide urea (PEAU), a leucine-based poly ester amide polymer, or another amino acid based copolymer. Due to both groups, ester and amide, such polymers are biodegradable (ester group) and have good thermal stability and mechanical strength (amide group with strong intermolecular interactions). The incorporation of leucine, or other suitable amino acid, improves the biocompatibility of the polymer. The biodegradation rate of this polymer can easily be adjusted by changing its exact composition and molecular weight. When capsules are formed, the liberation rate of any product incorporated therein can be adjusted by controlling the size and thickness of the capsules.

Such a polymer is synthesized, in some embodiments, by interfacial polycondensation of the monomer L6, di-p-sulfonic acid salt of bis-(L-leucine)-1,6-hexylene diester with trisphogene/sebacoyl chloride with water/dichloromethane system. This method is fast, irreversible, involves two immiscible phases at room temperature and lead to high molecular weight polymer. Synthesis of the monomer L6 was executed in the presence of p-toluene sulfonic acid by condensation of L-leucine with 1,6-hexanediol in refluxed cyclohexane, because it is less toxic than solvents such as benzene. Purification includes recrystallization from water, filtration and drying under vacuum.

The formulations containing microcapsules are fabricated using a water-in-oil-in-water double emulsion-solvent, for example using dichloromethane (DCM). The DCM can also be replaced by another suitable solvent, such as ethyl acetate, chloroform, or another organic solvent. In some embodiments, the double emulsion produces nanocapsules in a size range of approximately 100-400 nanometers, but other dimensions, as mentioned above, are also obtainable. One should note that since cannabinoids are lipophile, encapsulating them through a water-in-oil-in-water double emulsion technology seems at first counter-intuitive, and is not a direct application of this technique.

Generally speaking, polymers usable in the invention include: A polymer selected from
(1) a poly (ester amide urea) wherein at least one diol, at least one diacid, and at least one amino acid are linked together through an ester bond, an amide bond, and a urea bond,
(2) a poly (ester urethane urea) wherein at least one diol and at least one amino acid are linked together through an ester bond, a urethane bond, and a urea bond,
(3) a poly (ester amide urethane urea) wherein at least one diol, at least one diacid, and at least one amino acid are linked together through an ester bond, an amide bond, a urethane bond, and a urea bond,
(4) a poly (ester amide urethane) wherein at least one diol, at least one diacid, and at least one amino acid are linked together through an ester bond, an amide bond, and a urethane bond, (5) a poly (ester urea) wherein at least one diol and at least one amino acid are linked together through an ester bond and a urea bond, and
(6) a poly (ester urethane) wherein at least one diol and at least one amino acid are linked together through an ester bond and a urethane bond, further wherein
the at least one diol is a compound of formula:
   HO—R₁—OH, R₁ is chosen from C₂-C₁₂ alkylene optionally interrupted by at least one oxygen, C₃-C₈ cycloalkylene, C₃-C₁₀ cycloalkylalkylene,

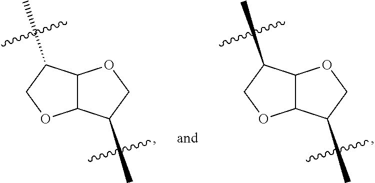

the at least one diacid is a compound of formula: HO—(CO)—R₃—(CO)—OH, R₃ is C₂-C₁₂ alkylene,
   the at least one amino acid is chosen from naturally occurring amino acids and non-naturally occurring amino acid.

In some embodiments, the polymer is selected from

(1) a poly (ester amide urea) wherein at least one diol, at least one diacid, and at least one amino acid are linked together through an ester bond, an amide bond, and a urea bond,
(2) a poly (ester urethane urea) wherein at least one diol and at least one amino acid are linked together through an ester bond, a urethane bond, and a urea bond,
(3) a poly (ester amide urethane urea) wherein at least one diol, at least one diacid, and at least one amino acid are linked together through an ester bond, an amide bond, a urethane bond, and a urea bond, and
(4) a poly (ester amide urethane) wherein at least one diol, at least one diacid, and at least one amino acid are linked together through an ester bond, an amide bond, and a urethane bond, wherein the at least one diol, at least one diacid, and at least one amino acid are as defined above.

In some more specific embodiments of the invention, the polymer is a poly (ester amide urea) comprising the following two blocks with random distribution thereof:

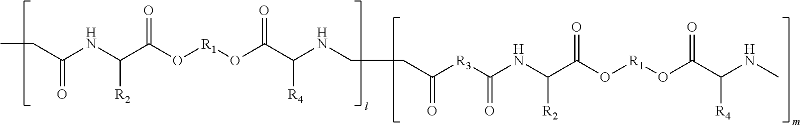

wherein

the ratio of 1:m ranges from 0.05:0.95 to 0.95:0.05, 1+m=1, R₁ is chosen from C₂-C₁₂ alkylenes optionally interrupted by at least one oxygen, C₃-C₈ cycloalkylenes, C₃-C₁₀ cycloalkylalkylenes,

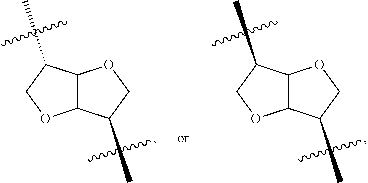

R₃ is C₂-C₁₂ alkylene,
R₂ and R₄ are independently chosen from the side chains of L- and D- amino acids so that the carbon to which R₂ or R₄ is attached has L or D chirality.

In some more specific embodiments of the invention, the polymer is poly (ester urethane urea) comprising the following two blocks with random distribution thereof:

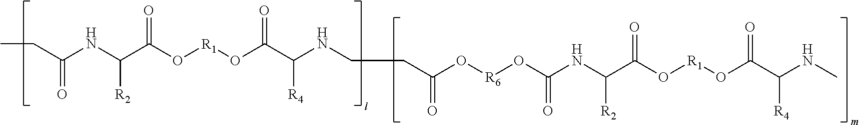

wherein
the ratio of 1:m ranges from 0.05:0.95 to 0.95:0.05, 1+m=1,
R₁ and R₅ are independently chosen from C₂-C₁₂ alkylenes optionally interrupted by at least one oxygen, C₃-C₈ cycloalkylenes, C₃-C₁₀ cycloalkylalkylenes,

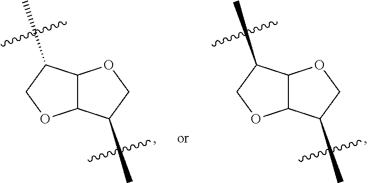

and
R₂ and R₄ are independently chosen from the side chains of L- and D- amino acids so that the carbon to which R₂ or R₄ is attached has L or D chirality.

In some more specific embodiments of the invention, the polymer is poly (ester amide urethane urea) comprising the following three blocks with random distribution thereof:

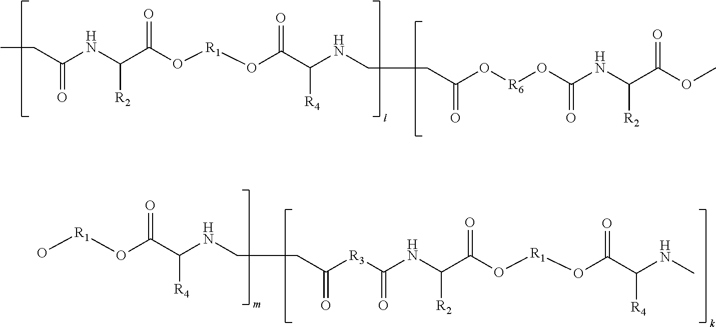

wherein
the ratio of 1:m:k ranges from 0.05:0.05:0.90 to 0.90:0.05:0.05, 1+m+k=1,
R₁ and R₅ are independently chosen from C₂-C₁₂ alkylenes optionally interrupted by at least one oxygen, C₃-C₈ cycloalkylenes, C₃-C₁₀ cycloalkylalkylenes,

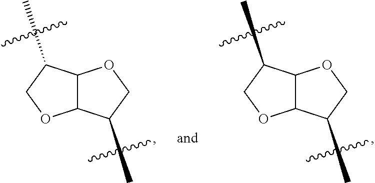

R₃ is C₂-C₁₂ alkylene, and
R₂ and R₄ are independently chosen from the side chains of L- and D- amino acids so that the carbon to which R₂ or R₄ is attached has L or D chirality.

In some more specific embodiments of the invention, the polymer is (ester amide urethane) comprising the following two blocks with random distribution thereof:

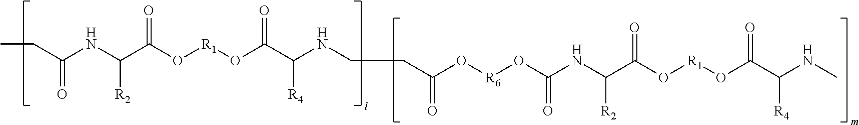

wherein

the ratio of 1:m ranges from 0.05:0.95 to 0.95:0.05, 1+m=1, R₁ and R₅ are independently chosen from C₂-C₁₂ alkylenes optionally interrupted by at least one oxygen, C₃-C₈ cycloalkylene, C₃-C₁₀ cycloalkylalkylene,

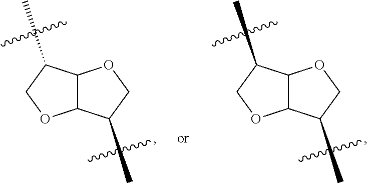

R₃ is C₂-C₁₂ alkylene, and
R₂ and R₄ are the same and selected from the side chains of L- and D- amino acids so that the carbon to which R₂ or R₄ is attached has L or D chirality.

In the above polymers, in some very specific embodiments of the invention, one or more of the following hold: R₁ is —(CH₂)₆—, R₃ is —(CH₂)₈—, or both R₂ and R₄ are the side chain of L-leucine.

Blends of the above-mentioned polymers are also usable in the preparation of the compositions of the present invention. More details regarding such polymers and others usable with the present invention are provided in PCT application PCT/US2016/038527 and U.S. patent application Ser. No. 15/188,783, the contents of which is hereby incorporated by reference in its entirety.

In some embodiments, inorganic particles are also encapsulated in the polymer capsules. Examples of the inorganic particles include particles of at least one salt selected from the group consisting of CaCO₃, Ca₃(PO₄)₂, MgCO₃, and Mg₃(PO₄)₂. Inorganic particles may be used to control the rate of biodegradation of the microcapsules by creating microchannels when the latter contact fluids.

In some embodiments, the ratio of 1:m ranges from 0.25:0.75 to 0.75:0.25, 1+m=1. In some embodiments, the amino-acid based polymer has a polydispersity of 1.15 or less. In some embodiments, the amino-acid based polymer has a molecular weight between 15 kDa and 90 kDa. In some embodiments, the amino-acid based polymer is amorphous. In some embodiments, the composition is powder form. In some embodiments, the composition is in liquid form, and in some specific embodiments, the polymer capsules are suspended in a solution including a poloxamer, for example poloxamer 407 in a concentration of between 10 and 30 percent. In some embodiments, the poloxamer has a mean molecular weight of between 9500 kDa and 15000 kDa.

In some embodiments, the composition wherein the composition is in gel form.

Generally speaking, the present compositions include polymer capsules in which cannabinoids are contained and are prepared by preparing a first aqueous phase; preparing a second aqueous phase; preparing an hydrophobic phase including an amino-acid based polymer dissolved therein; emulsifying the first aqueous phase in the hydrophobic phase to prepare a primary emulsion; and emulsifying the primary emulsion in the second aqueous phase to prepare a secondary emulsion. CBD loaded polymeric nanocapsules are prepared using a double emulsion, solvent evaporation process. The CBD solution, in the first aqueous phase, is emulsified into a polymer organic solution to form a water-in-oil (W/O) primary emulsion. The primary emulsion is added to an aqueous continuous phase to form a stable water-oil-water (W/O/W) double emulsion.

In some embodiments, the primary emulsion includes the first aqueous phase and the hydrophobic phase in a ratio of from 1:2 to 1:20. In some embodiments, the primary emulsion includes the first aqueous phase and the hydrophobic phase in a ratio of about 1:10. In some embodiments, the secondary emulsion includes the primary emulsion and the second aqueous phase in a ratio of from 1:2 to 1:20. In some embodiments, the secondary emulsion includes the primary emulsion and the second aqueous phase in a ratio of from 1:2 to 1:5.

In some embodiments, the preparation method further includes evaporating the organic solvent. In some embodiments, the organic solvent is evaporated without transfer to a hardening tank. For example, the organic solvent includes at least one of dichloromethane (DCM), ethylacetate and chloroform.

In some embodiments, emulsifying the first aqueous phase in the hydrophobic phase to prepare the primary emulsion includes stirring the first aqueous phase in the hydrophobic phase using an homogenizer operating a between 8000 RPM and 50000 RPM during preparation of the primary emulsion, for example at about 35000 RPM. In some embodiments, the homogenization of the primary emulsion lasts for 30 s or less.

In some embodiments, emulsifying the hydrophobic phase in the second aqueous phase to prepare the secondary emulsion includes adding the primary emulsion to the second aqueous phase while stirring the second aqueous phase using a homogenizer operating a between 8000 RPM and 25000 RPM, for example 35000 RPM. In some embodiments, homogenization lasts at least one of more than 1 min, more than 5 min and more than 10 min during preparation of the secondary emulsion.

In some embodiments, the hydrophobic phase includes from 5% to 20% w/v of polymer, for example about 12 to 13% w/v of polymer.

In some embodiments, the preparation method further including dialysing the secondary emulsion.

The invention may also provide a method wherein the first aqueous phase includes Kolliphor P188.

TIn some embodiments, the first aqueous phase includes polyvinyl alcohol, for example between 1% and 5% w/v of the polyvinyl alcohol and the polyvinyl alcohol has a mean molecular weight of between 10 kDa and 100 kDa. In some embodiments, the second aqueous phase also includes polyvinyl alcohol, for example between 1% and 5% w/v of the polyvinyl alcohol and the polyvinyl alcohol has a mean molecular weight of between 10 kDa and 100 kDa.

In some embodiments, wherein the amino-acid based polymer has a molecular weight between 15 kDa and 30 kDa.

In some embodiments, the preparation method further includes dissolving a gelling agent in the secondary emulsion, such as poloxamer 407 or any other suitable poloxamer, having for example a mean molecular weight of between 9500 kDa and 15000 kDa.

The example below use a polymer referred to as PEAU. This polymer is a poly (ester amide urea) comprising the following two blocks with random distribution thereof:

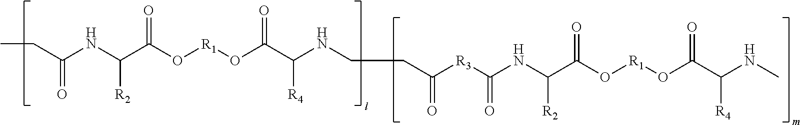

wherein
the ratio of 1:m ranges from 0.05:0.95 to 0.95:0.05, 1+m=1,
R₁ is chosen from C₂-C₁₂ alkylenes optionally interrupted by at least one oxygen, C₃-C₈ cycloalkylenes, C₃-C₁₀ cycloalkylalkylenes,

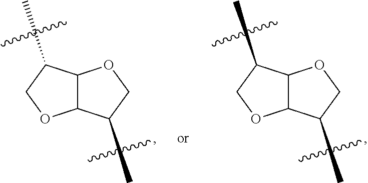

R₃ is C₂-C₁₂ alkylene,
R₂ and R₄ are independently chosen from the side chains of L- and D- amino acids so that the carbon to which R₂ or R₄ is attached has L or D chirality.

The more specific polymer referred to in the examples is the polymer wherein R₁ is —(CH₂)₆—, R₃ is —(CH₂)₈— and both R₂ and R₄ are the side chain of L-leucine. These polymers are referred to hereinbelow in abreviated form as (8L6)₁-(1L6)ₘ.

The following protocols are usable to manufacture the proposed composition. Cannabinoid loaded polymeric nanocapsules are prepared using a double emulsion, solvent evaporation process. The cannabinoid solution is emulsified into a polymer organic solution to form a water-in-oil (W/O) primary emulsion. The primary emulsion is added to an aqueous continuous phase to form a stable water-oil-water (W/O/W) double emulsion.

### Example 1

A first composition was obtained as follows.

First Emulsion:
Prepare 12 mL of 12.5% (W/V) PEAU polymer in DCM
Mix in an Eppendorf tube 915 μL CBD oil with 285 μL kolliphor P188 4.21% (Final concentration 1%)
In a 50 mL falcon tube mix the 12 mL 12.5% PEAU solution with the CBD oil and kolliphor P188 mix, Homogenize at 35 000 RPM for 30s in an ice bath.

Second Emulsion:
Before the first emulsion is done, prepare 30 mL of PVA 8-88 10% in a 100 mL beaker with a magnet stir bar, and prepare an ice bath.
When first emulsion is completed, immediately put the Beaker with PVA 10% in the ice bath and start the second emulsion by adding the first one and homogenize at 35 000 rpm for 10 min.
Let it stir over night for DCM evaporation (18h). Final volume after evaporation is 30 mL.
Transfer formulation in 50 mL falcon tube and store at 4° C.

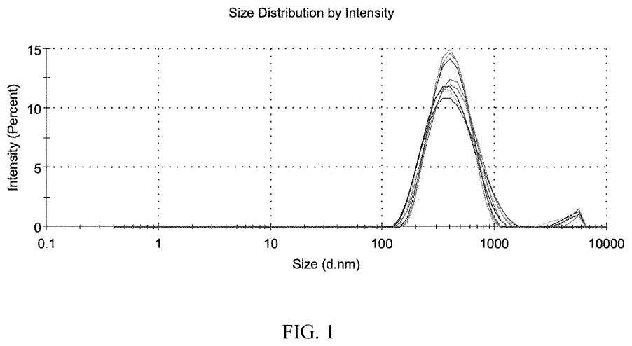

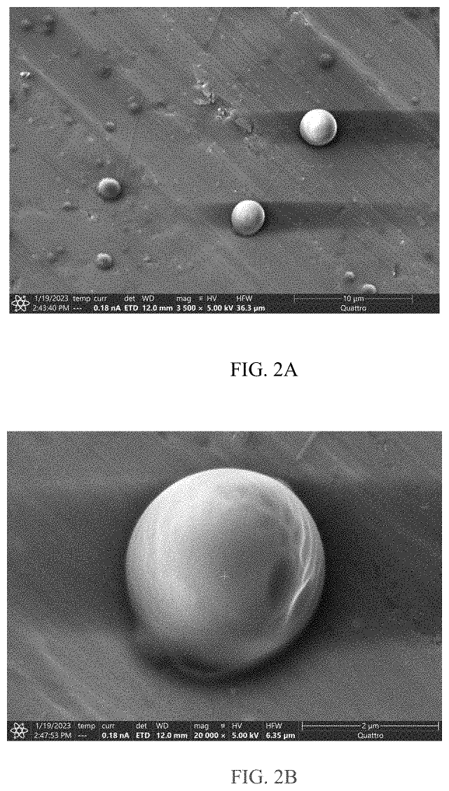

The particle size distribution of the capsules obtained in this example are shown in FIG. 1. FIGS. 2A and 2B show that the capsules are relatively smooth and are close to perfect spheres. It was also observed that the majority of the capsules were hollow. In other words, at least some of the CBD is present inside a hollow solid shell.

### Example 2

Tables 1 and 2 below summarize some characteristics of the formulation obtained with the examples 1 to 3. As see in Table 2, the Zeta potential of the formulation of example 1 was relatively low. Tween (TM) 80 was used to correct this problem. Thus, the above protocol can be changed as follows:

First Emulsion:
Prepare 12 mL of 12.5% (W/V) PEAU polymer in DCM
Mix in an Eppendorf tube 915 uL CBD oil with 285 uL kolliphor P188 4.21% (Final concentration 1%)
In a 50 mL falcon tube mix the 12 mL 12.5% PEAU solution with the CBD oil and kolliphor P188 mix, Homogenize at 35 000 RPM for 30s in an ice bath.

Second Emulsion:
Before the first emulsion is done, prepare 30 mL of PVA 8-88 10% +0.1% tween 80 in a 100 mL beaker with a magnet stirbar, and prepare an ice bath.
Before the first emultion is done, prepare 30 mL of PVA 8-88 10% +0.1% tween 80 in a 100 mL beaker with a magnet stirbar, and prepare an ice bath.
When first emultion is completed, immediately put the Beaker with PVA 10% in the ice bath, and start the second emultion by adding the first one and homogenize at 35 000 rpm for 10 min.
Let it stir over night for DCM evaporation (18h). Final volume after evaporation is 30 mL.
Transfer formulation in 50 mL falcon tube and store at 4° C.

### Example 3

To prevent agglomerations at higher Tween 80 concentrations, the protocol of example 2 can be changed as follows.

First Emulsion:
Prepare 12 mL of 12.5% (W/V) PEAU polymer in DCM
Mix in an Eppendorf tube 915 uL CBD oil with 285 uL kolliphor P188 4.21% (Final concentration 1%)
In a 50 mL falcon tube mix the 12 mL 12.5% PEAU solution with the CBD oil and kolliphor P188 mix, Homogenize at 35 000 RPM for 30s in an ice bath.

Second Emulsion:
Before the first emulsion is done, prepare 30 mL of PVA 8-88 10% +0.1% tween 80 in a 100 mL beaker with a magnet stirbar, and prepare an ice bath.
When first emulsion is completed, immediately put the Beaker with PVA 10% in the ice bath and start the second emulsion by adding the first one and homogenize at 35 000 rpm for 10 min.
Let it stir over night for DCM evaporation (18h). Final volume after evaporation is 30 mL.
Add 0.3 mL tween 80, let it stir until a homogenous solution is obtained
Transfer formulation in 50 mL falcon tube and store at 4° C.

### RESULTS FROM EXAMPLES 1 to 3

Liquid chromatography-mass spectrometry (LC-MS) (Agilent (TM) 1200 coupled to TOF MS 6224) was used to quantify the CBD inside the nanocapsules. Mobile phase A was H20+0.1% FA and Mobile phase B was ACN+0.1% FA. The column used was Poroshell EC-C18 4.6×100 mm. 20-30 mg of formulation to analyze was put in a tube, followed by addition of 200 uL H20 and mixing by vortex. Afterwards, centrifugation at 11 000 rpm for 5 min was performed, the supernatent was removes, and the whole dilution to centrigation sequence was repeated. After addition and mixing of 100 uL DCM, 100 uL MeOH and 900 uL (MeOH:CAN:H2O) 50:30:20, centrifugation at 13 000 RPM for 15 minutes was performed. 20 UL of the resulting composition was diluted in 500 uL (MeOH:CAN:H2O) 50:30:20 and LC-MS was performed on 1 μL of this dilution. The results showing encapsulation efficiency from these measures are shown in Table. 1

TABLE 1
Encapsulation efficiency (EE)

| | Theorical CBD | Experimental CBD | |
|---|---|---|---|
| | concentration | concentration | |
| | (ug/mg of | (ug/mg of | |
| Formulation | nanocapsules) | nanocapsules) | EE (%) |
| Example 1 | 117.81 | 60.49 ± 5.97 | 51.35 ± 5.07 |
| Example 2 | 98.52 | 40.53 ± 1.26 | 41.14 ± 1.27 |
| Example 3 | 95.50 | 45.89 ± 3.24 | 48.05 ± 3.40 |

CBD encapsulation efficiency (EE), based on the theorical concentration in ug/mg of nanocapsules, showed that the 2 best formulation (with the most CBD kept inside) are from examples 1 and 3, with no significative difference in their EE (respectively 51.35±5.07 and 48.05±3.40). V8.0 has the lowest EE (41.14±1.27) and concentration of CBD encapsulated (40.53±1.26 ug/mg of nanocapsules).

Zeta potential was determined using a Malvern (TM) NANO-ZS (TM). Samples where diluted 1/100 before analysis, because of the opacity of the formulation. Formulations of examples 1, 2 and 3 had Zeta potentials of respectively −10.05±0.50 mV, −17.13±0.21 and −34.4±0.32. The formulation of example 1 had small zeta potential (−10.05 mV), indicating that the formulation will not be stable for a long time because of nanocapsules agglomeration. Tween 80 as a non-ionic surfactant was added in examples 2 and 3 to correct this and improve the Zeta potential. The formulation of Example 2, with 0.1% Tween 80 added during the second emulsion, has a zeta potential of −17.13 mV, almost the double of that of the formulation of Example 1. Even better results were obtained with the formulation of example 3, by adding 1% (V/V) Tween 80 after the overnight evaporation, resulting in a Zeta potential of around −30 mV (see table 2). Thus, making this formulation stable for a long period of time, because nanocapsules agglomeration will be prevented.

CBD oil was therefore successfully encapsulated using the double emulsion nanoencapsulation method. In some embodiments, the capsules have an average diameter of from about 200 nm to about 1 μm, for example about 400 nm. Since CBD is a lipophilic compound, a stabilizing agent, Kolliphor P188, was used to create a homogenous phase to use for the first emulsion. To form relatively small particles, emulsification was performed for a relatively long time, at a large RPM in the presence of PVA in the second emulsion. The relatively small size of the capsules thus produced allow transdermal application.

Furthermore, when the target particle size is used, for example with an average of 400 nm in diameter, the formulation has improved safety as the nanocapsules size are too big to pass through the blood brain barrier (which requires below 100 nm in size), making it safer than others formulation, but small enough to migrate up to the dermis layer. There is no currently available nanoencapsulated CBD using polymer on the market for transdermal application. Also, CBD is notoriously difficult to encapsulate, and the present process and polymer has been shown to provide a relatively large encapsulation efficiency, while preserving the ability to provide transdermal delivery. With CBD concentration of 60.49±5.97 ug/mg and 45.89±3.24 ug/mg, the CBD encapsulation can deliver from about 5 to 12 times more CBD per mg than currently existing transdermal formulations. This is due in part to the double emulsion process.

Although the present invention has been described hereinabove by way of exemplary embodiments thereof, it will be readily appreciated that many modifications are possible in the exemplary embodiments without materially departing from the novel teachings and advantages of this invention. Accordingly, the scope of the claims should not be limited by the exemplary embodiments, but should be given the broadest interpretation consistent with the description as a whole. The present invention can also be modified, without departing from the spirit and nature of the subject invention as defined in the appended claims.

What is claimed is:

1. A method of delivering at least one cannabinoid to a subject, the method comprising applying on a skin of the subject a composition including biodegradable capsules, wherein the biodegradable capsules include a solution of the at least one cannabinoid encapsulated in a hydrophobic polymer.

2. The method as defined in claim 1, wherein the at least one cannabinoid is provided to treat a condition selected from the group consisting of seizures associated with Lennox-Gastaut syndrome or Dravet syndrome, arthritis, chronic pain, anorexia, side-effects of chemotherapy, multiple sclerosis, migraine, muscle spasms, schizophrenia, epilepsy, and a dermatological condition.

3. The method as defined in claim 2, wherein the dermatological condition is selected from acne vulgaris, dermatitis, psoriasis, hidradenitis suppurativa, Kaposi Sarcoma, and pruritus.

4. The method as defined in claim 1, wherein the at least one cannabidoid includes cannabidiol (CBD), and wherein the composition releases from about 5 mg to about 50 mg of CBD over a duration of between 12 and 72 hours.

5. The method as defined in claim 4, wherein 15 mg of CBD is released in about 48 hours.

6. The method as defined in claim 1, wherein at least 10% of the CBD contained in the composition is delivered transdermally.

7. A composition comprising biodegradable polymeric capsules, the biodegradable polymeric capsules including a first phase encapsulated in a second phase surrounding the first phase, wherein the first phase includes at least one cannabinoid and the second phase includes a biodegradable polymer.

8. The composition as defined in claim 7, wherein the biodegradable polymeric capsules include at least one of nanocapsules and microcapsules.

9. The composition as defined in claim 7, wherein the biodegradable polymer includes a polymer selected from
(1) a poly (ester amide urea) wherein at least one diol, at least one diacid, and at least one amino acid are linked together through an ester bond, an amide bond, and a urea bond,
(2) a poly (ester urethane urea) wherein at least one diol and at least one amino acid are linked together through an ester bond, a urethane bond, and a urea bond,
(3) a poly (ester amide urethane urea) wherein at least one diol, at least one diacid, and at least one amino acid are linked together through an ester bond, an amide bond, a urethane bond, and a urea bond,
(4) a poly (ester amide urethane) wherein at least one diol, at least one diacid, and at least one amino acid are linked together through an ester bond, an amide bond, and a urethane bond,
(5) a poly (ester urea) wherein at least one diol and at least one amino acid are linked together through an ester bond and a urea bond, and
(6) a poly (ester urethane) wherein at least one diol and at least one amino acid are linked together through an ester bond and a urethane bond,
further wherein
the at least one diol is a compound of formula:
HO—R₁—OH, R₁ is chosen from C₂-C₁₂ alkylene optionally interrupted by at least one oxygen, C₃-C₈ cycloalkylene, C₃-C₁₀ cycloalkylalkylene, 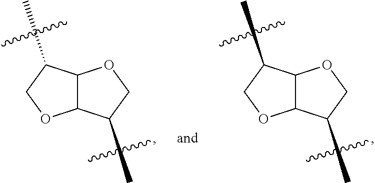
the at least one diacid is a compound of formula:
HO—(CO)—R₃—(CO)—OH, R₃ is C₂-C₁₂ alkylene,
the at least one amino acid is chosen from naturally occurring amino acids and non-naturally occurring amino acid.

10. The composition as defined in claim 9, wherein the polymer is a poly (ester amide urea) comprising the following two blocks with random distribution thereof: 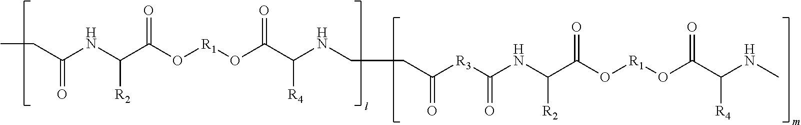
wherein
the ratio of 1:m ranges from 0.05:0.95 to 0.95:0.05, 1+m=1,
R₁ is chosen from C₂-C₁₂ alkylenes optionally interrupted by at least one oxygen, C₃-C₈ cycloalkylenes, C₃-C₁₀ cycloalkylalkylenes, 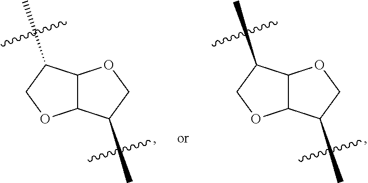
R₃ is C₂-C₁₂ alkylene,
R₂ and R₄ are independently chosen from the side chains of L- and D- amino acids so that the carbon to which R₂ or R₄ is attached has L or D chirality.

11. The composition as defined in claim 10, wherein R₁ is —(CH₂)₆—, R₃ is —(CH₂)₈—, and both R₂ and R₄ are the side chain of L-leucine.

12. The composition as defined in claim 7, further comprising a permeation enhancer.

13. The composition as defined in claim 12, wherein the permeation enhancer is selected from the group consisting of hydrocarbons, including alkanes, alkenes, squalene, mineral oil, halogens, alcohols, including glycerols, glycols, polyglycols, ethanol, caprylic alcohol, laury alcohol, organic acids, including oleic acid, undecanoic acid, amines, amides, including pyrrolidone(N-methyl-2-pyrrolidone 2-pyrrolidon)azones, (1-dodecylazacycloheptan-2-one))urea, isopropyl myristate, sodium lauryl sulfate, cetyltrimethyl ammonium bromide, sorbitan monolaurate, polysorbate 80, dodecyl dimethyl ammoniopropane sulfate, dimethyl sulfoxide, dodecyl methyl sulfoxide, phospholipids, water (for example 70:30, v/v), and carbopol.

14. The composition as defined in claim 10, wherein the formulation is for administration across at least one of dermal and epidermal barriers, and wherein the capsules are configured so as to not enter systemic circulation.

15. The composition as defined in claim 7, wherein the at least one cannabinoid is derived from at least one of cannabis concentrates, isolated cannabinoids and cannabis extracts.

16. The composition as defined in claim 7, wherein the at least one cannabinoid is selected from the group consisting of: phytocannabinoids, synthetic cannabiboids, and cannabimimetics.

17. The composition as defined in claim 7, further comprising hyaluronic acid.

18. The composition as defined in claim 7, further comprising collagen.

19. The composition as defined in claim 7, wherein the at least one cannabinoid is cannabigerol (CBG).

20. The composition as defined in claim 7, wherein the at least one cannabinoid is cannabidiol (CBD).

21. The composition as defined in claim 7, wherein the capsules include nanocapsules having an outer diameter of between 10 nm and 1 μm.

22. The composition as defined in claim 21, wherein the nanocapsules are between 100 nm and 1 μm in diameter.

23. The composition as defined in claim 7, wherein the capsules include microparticles having an outer diameter of between 1 μm and 250 μm.

24. The composition as defined in claim 7, wherein the first phase includes the at least one cannabinoid in a solution, the solution including the at least one cannabinoid, a solvent and an emulsifier.

25. The composition as defined in claim 24, wherein the emulsifier includes Kolliphor P188.

26. The composition as defined in claim 24, wherein the solvent is an oil.

27. The composition as defined in claim 7, further comprising Tween 80.
